(19) United States
(12) Patent Application Publication
(10) Pub. No.: US 2022/0195069 A1
(43) Pub. Date: Jun. 23, 2022

(54) PCSK9 INHIBITORS FOR NEUROPATHY

(71) Applicant: MANCHESTER UNIVERSITY NHS FOUNDATION TRUST, Manchester (GB)

(72) Inventor: Handrean SORAN, Manchester (GB)

(21) Appl. No.: 17/594,648

(22) PCT Filed: Apr. 27, 2020
(86) PCT No.: PCT/EP2020/061637

§ 371 Date: Oct. 25, 2021

(30) Foreign Application Priority Data
Apr. 25, 2019 (GB) 1905779.3

(51) Int. Cl. C07K 16/40; G01N 33/68; A61P 25/02

(52) U.S. Cl. CPC C07K 16/40; A61K 2039/505; A61P 25/02; G01N 33/6896

(57) ABSTRACT

The present invention relates to an agent for use in a method of treatment of neuropathy in a subject in need thereof. The invention also relates to a method of diagnosing neuropathy in a subject, based on detecting a level of free or total PCSK9 in a sample of blood, such as a serum sample.

### FIELD

The present invention relates to an agent for use in a method of treatment of neuropathy in a subject in need thereof. The invention also relates to a method of diagnosing neuropathy in a subject.

### BACKGROUND

Neuronal damage to the peripheral nerves, commonly termed peripheral neuropathy, causes significant morbidity to the adult population. It is estimated that nearly 1 in 10 people in the UK have some form of peripheral neuropathy. This is particularly prevalent in diabetic subjects, with approximately 60 to 70% of diabetic patients developing neuropathy. This can result in ulcers, infections, amputations and even death of the diabetic patient.

Current treatment options for peripheral neuropathy, particularly diabetic neuropathy, are limited. Treatment often involves medication to relieve nerve pain, such as pregabalin. However, this does not treat the underlying cause of the neuropathy, or treat the loss of sensation which can lead to ulcers, infections and amputations. In addition, none of the current therapies are known to regenerate nerve fibres.

Diagnosis of diabetic neuropathy can also be difficult. Although numerous tests exist, these are often qualitative, time consuming and/or invasive. In addition, known tests generally rely on the presentation of symptoms by the subject. Yet many diabetic patients have nerve damage but are initially asymptomatic.

Proprotein convertase subtilisin/kexin type 9 (PCSK9) is a protein produced by mainly hepatocytes but expressed in many other tissues. PCSK9 binds to low-density lipoprotein particles receptor (LDLR). Once the receptor binds LDL particle, it is internalised by the hepatocyte cell. Binding of PCSK9 to the receptor initiates a conformational change in the receptor. This leads to re-direction of the LDL receptor, once internalised, to the lysosome rather than being recycled back to the cell surface. Recent studies have shown that PCSK9 inhibitors are attractive therapeutic targets for lowering LDL-cholesterol.

The present invention has been devised with these issues in mind.

### SUMMARY OF THE INVENTION

At its broadest, the present disclosure relates to PCSK9 as a biomarker for neuropathy and as a treatment target for neuropathy. The PCSK9 biomarker can be used in the diagnosis and/or target for treatment of neuropathy.

According to a first aspect, there is provided an agent for use in a method of treatment of peripheral and autonomic neuropathies in a subject in need thereof, wherein the agent is capable of inhibiting PCSK9.

By “capable of inhibiting PCSK9”, it will be understood that the agent functions to decrease the activity of PCSK9, or the level of expression of PCSK9, relative to normal levels (i.e. activity or expression levels in the absence of the agent). The level of expression may be of the PCSK9 gene or of the gene product, for example PCSK9 mRNA or protein. In one embodiment, the agent functions to decrease the activity of PCSK9

In some embodiments, the “activity of PCSK9” may be understood to refer to the binding of PCSK9 to the LDL receptor and/or an LDL particle. However, in some embodiments an agent may inhibit PCSK9 in a manner which does not, or not only, acts through inhibiting the binding of PCSK9 to the LDL receptor and/or LDL particle.

The agent may be capable of directly or indirectly inhibiting PCSK9. For example, the agent may be a nucleic acid that specifically binds to PCSK9 mRNA, thereby causing direct repression of expression of the PCSK9 gene into a protein (and thus indirectly decreasing PCSK9 protein activity). In another example, the agent may be an antibody or antibody fragment that specifically binds to PCSK9 protein, thereby causing direct repression of the activity of the PCSK9 protein. In another example, the agent may be a small molecule which indirectly causes PCSK9 gene expression to be decreased through activation of a transcriptional repressor, or by affecting post-translational modifications.

In some embodiments the agent decreases the expression or activity of PCSK9 by at least 20%, at least 30%, at least 40%, at least 50%, at least 70%, at least 80%, at least 90%, at least 95% or substantially 100%.

The activity of PCSK9 can be measured in vitro using assays known to the skilled person, for example using protein binding or a FRET (forster resonance energy transfer) assay.

The agent may comprise or consist of a peptide, a protein, an enzyme, an antibody, a nucleic acid (e.g. a siNA, a ribozyme or a plasmid), a bacteriophage, a plant alkaloid or a small molecule.

In some embodiments the agent is a naturally-occurring or a synthetic molecule.

As used herein, a “small molecule” is a chemical compound having a molecular weight of no more than 2000 daltons (Da). In some embodiments, the small molecule has a molecular weight of no more than 1000, such as no more than 700 or no more than 500 Da. The small molecule may be an organic compound. Small molecule inhibitors are described, for example, in Shengtao et al., European Journal of Medicinal Chemistry, 162, 2019, p 212-233, the entire contents of which are hereby incorporated by way of reference.

A bacteriophage may comprise a DNA or RNA sequence encoding a peptide epitope of PCSK9. In vivo, the peptide epitope of PCSK9 is expressed from the bacteriophage. This causes antibodies specific for PCSK9 to be raised against the peptide epitope in vivo.

An exemplary peptide PCSK9 inhibitor is described in Shan et al., Biochem. Biophys. Res. Commun. 375 (1): 69-73, the entire contents of which are hereby incorporated by way of reference.

In some embodiments the agent is a plant alkaloid, for example berberine.

In some embodiments the agent comprises or consists of an antisense molecule (e.g. an antisense DNA or RNA molecule or a chemical analogue) or a ribozyme molecule.

Ribozymes may be used to inhibit the translation of mRNA encoding PCSK9 protein. Ribozymes are RNA molecules capable of exhibiting enzymatic activity.

Antisense molecules may be used to inhibit the transcription of a gene encoding PCSK9 or inhibit translation of the PCSK9 mRNA of that gene. Antisense molecules are oligonucleotides that bind in a sequence-specific manner to nucleic acids, such as DNA or RNA. When bound to mRNA that has a complementary sequence, antisense RNA prevents translation of the mRNA. Triplex molecules refer to single stranded antisense DNA strands that bind duplex DNA forming a colinear triplex molecule, thereby preventing transcription. Particularly useful antisense molecules and triplex molecules are ones that are complementary to or bind the sense strand of DNA (or mRNA) that encodes PCSK9 protein.

In some embodiments, the inhibitor comprises or consists of miRNA (microRNA), or shRNA (short hairpin RNA).

In some embodiments, the agent comprises or consists of a short interfering nucleic acid (siNA). Oligonucleotides including siNAs can be prepared by solid phase chemical synthesis using standard techniques. A siNA molecule may comprise a siDNA molecule or a siRNA molecule. In some embodiments, the inhibitor is a siRNA. An exemplary siRNA molecule is Inclisiran marketed by the Medicines Company.

In some embodiments, the agent comprises or consists of a CRISPR knockout product. CRISPR knockout products, such as CRISPR/Cas9 knock-out plasmids, are commercially available and enable the identification and cleavage of a gene of interest, thereby eliminating production of the gene product.

In some embodiments, the agent comprises a peptide, a protein, an enzyme or an antibody. Proteins and peptides may be generated using a variety of methods, including purification of naturally-occurring proteins, recombinant protein production and de novo chemical synthesis. Methods for generating antibodies are well-known to those skilled in the art.

It will be understood that the term “antibody”, as used herein, includes any form of antibodies, including domain antibodies, Fc, Fab and F(ab′)2 fragments, single chain variable region fragments (scFV) or IgA/B/C/D/E/F and especially IgG (any of subtypes 1, 2 3 or 4). Where reference is made herein to an antibody, it will be appreciated that this includes biopharmaceuticals. These biopharmaceuticals may include humanised antibodies, domains and fragments of antibodies, chimeric antibodies, bi-specific antibodies, antibody-drug conjugates, non-immunoglobulin protein scaffolds including, but not restricted to adnexins, darpins, camelids, shark variable domains and non-protein domains including but not restricted to aptamers.

The antibody may be human or humanised versions of any of the antibodies described herein. If that reference antibody is not human or humanised, then a humanised or human version thereof is preferred. The generation of humanised or human antibody from, for example, murine antibody is generally well known in the art.

In some embodiments the agent comprises or consists of an antibody, for example a monoclonal antibody. The monoclonal antibody may be any one of evolocumab (Amgen), bococizumab (Pfizer), 1D05-IgG2 (Merck), RG-7652 and LY3015014, and alirocumab (Aventis/Regeneron), all of which are readily commercially available and are known in the art. In some embodiments the antibody is evolocumab or alirocumab. In some embodiments the antibody is evolocumab.

It will be understood that the term “treatment”, as used herein, includes the reduction, alleviation, and/or removal of symptoms of neuropathy, the delay of progression of neuropathy, and/or substantially curing of neuropathy in the subject.

Neuropathy is a known term to describe neuronal damage. In the context of the present invention, neuropathy is used to define neuronal damage of the peripheral nerves, i.e. nerves other than the brain and spinal cord.

Symptoms of neuropathy may include one or more of cramp, fasciculation (fine muscle twitching), tingling in the hands and/or feet, pain, muscle loss, muscle weakness, bone degeneration, impaired balance and/or coordination and numbness.

Neuropathy is classified based upon the type and number of nerves affected by the damage. An example neuropathy, mononeuropathy, is a type of neuropathy that affects a single nerve. Generally, this is caused by localised trauma, for example compression or an infection to a nerve. Another example neuropathy, polyneuropathy relates to nerve damage in a plurality of nerves.

Types of neuropathy classified by the type of nerve affected include motor neuropathy, sensory neuropathy and autonomic neuropathy. In some embodiments the neuropathy comprises or consists of motor neuropathy, sensory neuropathy and/or autonomic neuropathy. Motor neuropathy defines damage of the motor neurons, sensory neuropathy defines damage of the sensory neurons and autonomic neuropathy defines damage of the autonomic neurons.

Symptoms of motor neuropathy may include one or more of muscle weakness, impaired balance and/or coordination, fasciculation and muscle paralysis.

Sensory neuropathy may include one or more of the following symptoms: numbness, reduced ability to sense pain or extreme temperatures, tingling in the hands and/or feet, burning sensation (dysesthesia) and pain.

The symptoms of autonomic neuropathy depend on the area of the body affected. For example, the heart rate, blood pressure, digestive system, bladder, sex organs, sweat glands and/or eyes may be affected.

In some embodiments the neuropathy comprises sensory neuropathy. The neuropathy may comprise small fibre neuropathy, a type of sensory neuropathy.

The neuropathy may comprise or consist of small fibre neuropathy and/or large fibre neuropathy. Small fibre neuropathy is characterised by damage of the small fibres of the peripheral nervous system, for example damage to C fibres and small AO fibres. Large fibre neuropathy is characterised by damage of the large fibres of the peripheral nervous system.

In some embodiments the neuropathy comprises retinopathy. The neuropathy may comprise retinopathy and small fibre neuropathy.

The neuropathy may be diabetic neuropathy. In some embodiments the subject has type 1 or type 2 diabetes, preferably type 2 diabetes. In some embodiments the subject has diabetes secondary to pancreatitis, diabetes secondary to a medication which increases insulin resistance, monogenic diabetes and/or latent autoimmune diabetes in adults. In some embodiments the subject may display obesity or familial hypercholesterolemia (FH) with no associated diabetes at the time of treatment.

A number of methods are available for diagnosing neuropathy, as will be known to those skilled in the art. Typical tests for the diagnosis of neuropathy include the Neuropathy Symptom Profile (NSP), the modified Neuropathy Disability Score (NDS), the Vibration perception threshold (VPT), cold (CT) and warm (WT), sural sensory nerve amplitude, conduction velocity and latency and peroneal motor nerve amplitude, conduction velocity and latency, heart rate variability to deep breathing (HRV-DB) and skin biopsy.

The subject may be one who has already been diagnosed as having neuropathy by a clinician. In some embodiments, the neuropathy was previously diagnosed by at least one of the Neuropathy Symptom Profile (NSP), the modified Neuropathy Disability Score (NDS), the Vibration perception threshold (VPT), cold (CT) and warm (WT), sural sensory nerve amplitude, conduction velocity and latency and peroneal motor nerve amplitude, conduction velocity latency, heart rate variability to deep breathing (HRV-DB) and skin biopsy.

Corneal confocal microscopy (CCM) can be used to identify neuronal damage in the corneal tissue. Advantageously, CCM is non-invasive. Results from CCM can also be obtained more quickly than some other neuropathy diagnosis methods.

Thus, in some embodiments the neuropathy was previously diagnosed by corneal confocal microscopy. CCM may be used to assess corneal nerve fibre density (CNFD), corneal nerve fibre length (CNFL) and/or corneal nerve branch density (CNBD) in the subject.

Neuropathy may be diagnosed when the CNFD, CNBD and/or CNFL is reduced relative to a respective reference level of CNFD, CNBD and/or CNFL. The reference level may be, for example, the CNFD, CNBD and/or CNFL determined in a healthy control subject without neuropathy, or a “normal” value of CNFD, CNBD and/or CNFL reported in the literature. Thus, the CNFL, CNFD and/or CNBD in the subject may be reduced by at least 10%, 20%, 50%, 70%, 90% or 100% relative to the respective reference level.

Without wishing to be bound by theory, the present inventor believe that the invention may be of particular benefit to overweight or obese subjects.

The standard method of the art for determining whether or not a subject is overweight or obese is the Body Mass Index (BMI) method. A BMI of 18.5 to 24.9 is considered to mean the subject is a healthy weight. A BMI of 25 to 29.9 means the subject is overweight. A BMI of 30 to 39.9 means the subject is obese, while a BMI of 40 or above means the subject is morbidly obese.

Thus, in some embodiments, the subject is overweight, i.e. has a BMI of 25 or more. The subject may be obese, i.e. the subject may have a BMI of 30 or more, for example 30 to 39.9. The subject may have a BMI of 40 or more.

In some embodiments, the subject is a mammal. In some embodiments, the subject is human. Non-human subjects to which the invention is applicable include pets, domestic animals, wildlife and livestock, including dogs, cats, cattle, horses, sheep, goats, deer and rodents.

Administration of the agent may be by any suitable route, including but not limited to, injection (including intravenous (bolus or infusion), intra-arterial, intraperitoneal, subcutaneous (bolus or infusion), intraventricular, intramuscular, or subarachnoidal), oral ingestion, inhalation, topical, via a mucosa (such as the oral, nasal or rectal mucosa), by delivery in the form of a spray, tablet, transdermal patch, subcutaneous implant or in the form of a suppository.

In some embodiments administration of the agent to the subject is by injection, preferably intravenous, intramuscular or intradermal injection.

The agent may be administered in the form of a composition. The composition may further comprise a pharmaceutically acceptable carrier. A “pharmaceutically acceptable carrier” as referred to herein is any physiological vehicle known to those of ordinary skill in the art useful in formulating pharmaceutical compositions. The agent may be mixed with, or dissolved, suspended or dispersed in the carrier.

The composition may be in the form of a capsule, tablet, liquid, ointment, cream, gel, hydrogel, aerosol, spray, micelle, transdermal patch, liposome or any other suitable form that may be administered to a subject suffering from neuropathy.

In embodiments wherein the agent is a peptide or protein, a nucleic acid sequence encoding the peptide or protein may be provided in a suitable vector, for example a plasmid, a cosmid or a viral vector. Thus, also provided is a vector (i.e. a construct), comprising a nucleic acid sequence which encodes the protein or peptide. The nucleic acid sequence is preferably operably linked to a suitable promoter. The invention further relates to a composition comprising the vector.

Inhibitors which are nucleic acids, such as siRNAs or miRNAs, may be modified (e.g. via chemical modification of the nucleic acid backbone, for example PEGylation) to improve the stability of the nucleic acid. This may protect the nucleic acid from degradation. The nucleic acid may be delivered in a suitable delivery system which protects the nucleic acids from degradation and/or immune system recognition. Examples of suitable delivery systems include nanoparticles, lipid particles, polymer-mediated delivery systems, lipid-based nanovectors and exosomes.

In some embodiments, the method comprises administering a dose of between 50 and 1000 mg of the agent to the subject, preferably a dose of between 130 and 450 mg. The method may comprise administering a dose of 140 mg of the agent to the subject.

The method may comprise administration of a single dose or a multiple dose of the agent. Multiple doses may be administered in a single day (e.g. 2, 3 or 4 doses at intervals of e.g. 3, 6 or 8 hours). The method may comprise administration of the agent on a regular basis (e.g. daily, every other day, weekly, fortnightly or monthly) over a period of days, weeks or months, as appropriate.

The dosage regime of the agent may be modified to take into account the diurnal rhythm of PCSK9 expression. Thus, in some embodiments the method comprises administering the agent at a time of day at which peak expression of PCSK9 is expected or determined to occur.

In some embodiments the method comprises administering the agent to the subject once a fortnight. The agent may be administered once a month. The total treatment duration may be one, two, three, six or more months.

It will be appreciated that optimal doses to be administered can be determined by those skilled in the art, and will vary depending on the particular inhibitor in use, the strength of the preparation, the mode of administration, the advancement or severity and type of the neuropathy. Additional factors depending on the particular subject being treated will result in a need to adjust dosages, including subject age, weight, gender, diet, and time of administration. Known procedures, such as those conventionally employed by the pharmaceutical industry (e.g. in vivo experimentation, clinical trials, etc.), may be used to establish specific formulations for use according to the invention and precise therapeutic dosage regimes.

In some embodiments the agent is administered with a further therapeutic agent, for example a LDL cholesterol lowering medication. Thus, the invention further provides a combination therapy for use in a method of treating neuropathy in a subject in need thereof.

The agent and the further therapeutic agent may be administered to the subject sequentially, simultaneously or separately.

The subject may be a subject previously treated with an LDL cholesterol lowering medication or a subject already taking an LDL cholesterol lowering medication.

Example LDL-lowering cholesterol medications include but are not limited to statins, niacin, fibrates, Ezetimibe, fish oil based therapies and bile acid sequestrants. Example fish oil based therapies include, but are not limited to, Omacor, Vascepa and Prestylon.

In some embodiments the fibrate comprises fenofibrate or gemfibrozil. Suitable bile acid sequestrants include cholestyramine, colesevelam and colestipol.

The LDL-lowering cholesterol medication may comprise a statin, for example atorvastatin, cerivastatin, fluvastatin, lovastatin, mevastatin, pitavastatin, pravastatin, rosuvastatin, or simvastatin or a combination thereof.

Administration of the agent to the subject may cause an increase in the CNFD, CNFL and/or CNBD in the subject. The CNFL, CNBD, and/or CNFD may be determined, for example by cornea confocal microscopy.

Thus, in some embodiments, the method further comprises determining the CNFD, CNFL and/or CNBD in the subject after treatment, and comparing the CNFD, CNFL and/or CNBD so determined to the CNFD, CNFL and/or CNBD (respectively) determined in the subject prior to treatment (i.e. prior to administering the agent). If the CNFD, CNFL and/or CNBD following treatment is greater than before treatment, this may be indicative of the success of the treatment. In such embodiments, the dosage of the agent administered may be reduced or stopped.

According to a further aspect there is provided a method of treatment of neuropathy, the method comprising administering a therapeutically effective amount of an agent to a subject in need thereof, wherein the agent is capable of inhibiting PCSK9.

As used herein, a “therapeutically effective amount” is an amount of the agent according to the first aspect of the invention which, when administered to a subject, is sufficient to eliminate, reduce or prevent neuropathy. A therapeutically effective amount may also be an amount at which there are no toxic or detrimental effects, or a level at which any toxic or detrimental effects are outweighed by the therapeutic benefits.

According to another aspect there is provided a method of diagnosing neuropathy in a subject, the method comprising:

(a) determining a level of PCSK9 expression in a sample obtained from a subject;

(b) comparing the determined level of PCSK9 expression with a reference level; wherein

the subject is diagnosed with neuropathy if the level of PCSK9 expression in the sample is higher than the reference level.

In the context of the present invention, reference level will be understood to mean a reference level of the respective parameter. For example, the CNFD of the subject would be compared to the CNFD reference level. The level of PSCK9 expression in the subject would be compared to the PCSK9 reference level.

A reference level may be obtained from a healthy control subject without neuropathy, or it may be an average level from a plurality of healthy control subjects without neuropathy, or a “normal” value reported in the literature.

Optionally, the method further comprises obtaining the sample from the subject.

In some embodiments the step of determining a level of PCSK9 expression in a sample obtained from a subject comprises determining a level of PCSK9 protein in the sample. The PCSK9 expression may be PCSK9 mRNA. In some embodiments both PSCK9 protein and mRNA levels are determined. In some embodiments, the level of PSCK9 is a free or total protein level, such as the free or total serum level. The skilled reader understands that the “free” level refers to PCSK9 when unbound to another molecule and the “total” level refers to both bound (i.e. bound to another molecule) and unbound PCSK9.

The present inventor has observed that there is an inverse correlation between certain nerve cell parameters, such as nerve fibre density, nerve branch density and/or nerve fibre length, and total or free serum PCSK9 levels. Moreover, the correlation between free PCSK9 and such nerve parameters, points to a direct involvement of PCSK9 in nerve damage rather than simply operating via LDL particles and LDL cholesterol. This may suggest a new mode of action in terms of treating neuropathies. Further, the inventor has observed a higher serum PCSK9 concentration in obese patients with evidence of small fibre neuropathy compared to similarly obese patients with no evidence of neuropathy.

mRNA levels may be determined using standard techniques known to those skilled in the art, for example qPCR. Protein levels may be determined using standard techniques known to those skilled in the art, for example Western Blot or immunofluorescence techniques.

The sample may comprise at least one of: urine, saliva, sputum, semen, mucus, tears, a vaginal swab, a rectal swab, a cervical smear, a tissue biopsy, a urethral swab blood and blood fractions such as plasma, serum. In some embodiments the sample is a fluid sample. Suitably, the fluid sample is one that can be readily obtained from the subject, such as urine, saliva, blood and sputum, which the individual may be able to collect from him/herself, without the need for assistance. In some embodiments the fluid sample is blood or a blood fraction such as plasma or serum.

In some embodiments the method further comprises determining the CNFL, CNBD, and/or CNFD in a CCM image obtained from the subject and comparing the determined CNFL, CNBD and/or CNFD with a respective reference level. The subject may be diagnosed with neuropathy if the level of PCSK9 expression in the sample is increased and the CNFL, CNBD and/or CNFD is decreased relative to the respective reference level.

Optionally, the method further comprises obtaining a CCM image of the cornea from the subject.

In some embodiments CNFL is determined in a CCM image obtained from the subject.

The CNFL, CNBD and/or CNFD may be reduced by at least 10, 20, 30, 40, 50, 60, 70, 90 or 100% relative to the respective reference level. In some embodiments the CNFL, CNBD and/or CNFD may be reduced by no more than 70, 60 or 50% relative to the respective reference level.

In some embodiments the method comprises determining CNFL, CNBD and/or CNFD from at least two, three, four, five, six, ten or twelve CCM images obtained from the subject.

The reference level may be an average (i.e. mean) of CNFL, CNBD and/or CNFD values determined from at least two, three, four, five, six, ten or twelve CCM images obtained from one or more healthy control subjects.

By providing a plurality of images, mean and standard deviation values can be calculated. The standard deviation of the reference level can advantageously be used to determine whether or not the CNFL, CNBD and/or CNFD is sufficiently lower to diagnose neuropathy. For example, in some embodiments the subject is diagnosed with neuropathy if the level of PCSK9 expression in the sample is increased relative to the respective reference level and the CNFL, CNBD and/or CNFD is decreased by at least two standard deviations relative to the respective reference level, the standard deviation used being that of the respective reference level.

By diagnosing the subject with neuropathy if the level of PCSK9 expression in the sample is increased relative to the respective reference level, and the CNFL, CNBD and/or CNFD is decreased relative to the respective reference level, this provides an accurate and fast test for the diagnosis of neuropathy without the need for numerous time-consuming, inaccurate and invasive tests.

In some embodiments the method further comprises administering a therapeutically effective amount of an agent as defined in the first aspect to a subject diagnosed as having neuropathy.

In another aspect there is provided a method of detecting a level of PCSK9 expression in a subject in order to detect whether or not a subject is suffering from a neuropathy, such as small fibre neuropathy, the method comprising:

determining a level of PCSK9 expression in a sample obtained from the subject, wherein the level of PCSK9 is total or free serum PCSK9 and an elevated level of PCSK9 is correlated with the subject suffering from the neuropathy.

The term neuropathy is to be understood as defined herein above.

Optionally, the method further comprises comparing the level of PCSK9 expression so determined with a reference PSCK9 level.

Optionally, the method further comprises obtaining the sample from the subject.

All of the features described herein (including any accompanying claims, abstract and drawings) may be combined with any of the above aspects in any combination, unless otherwise indicated.

### DETAILED DESCRIPTION

The invention will now be described with reference to the accompanying Figures and Tables in which:

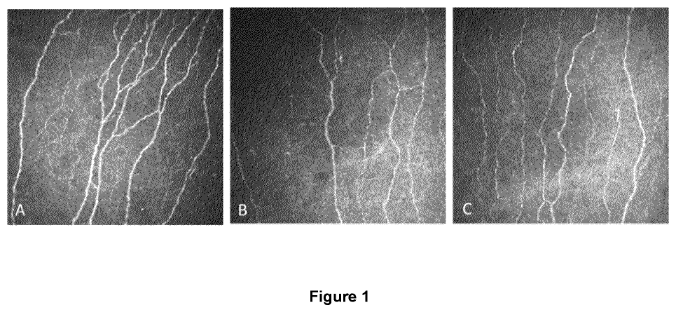

FIG. 1 shows photographs of a cornea performed with a corneal confocal microscope (CCM) graph showing changes in corneal nerves in subjects with heterozygous familial hypercholesterolemia (HeFH) prior to treatment (B: baseline) and six months after treatment with a PCSK9 monoclonal antibody (C: 6 months) compared to age and sex matched healthy control subjects (A);

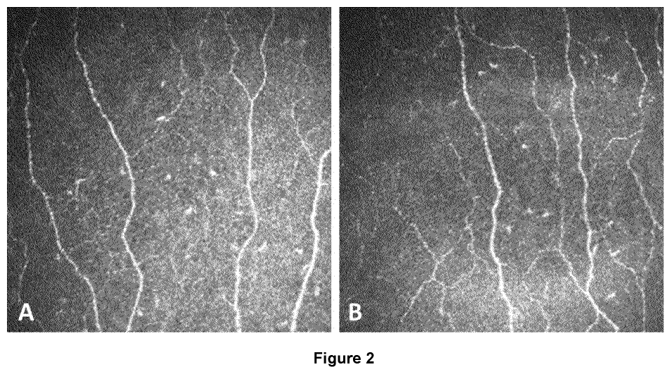

FIG. 2 CCM images from a diabetic patient at baseline (A) and 6 months after treatment with a PCSK9 monoclonal antibody (B);

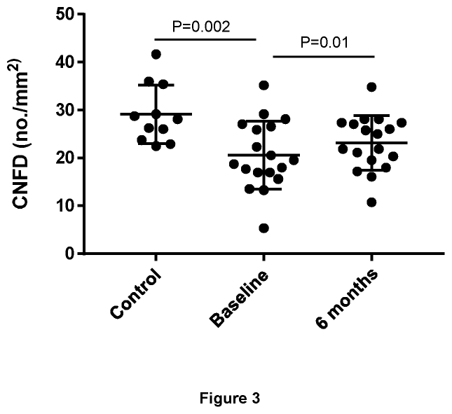

FIG. 3 is a graph showing changes in corneal nerve fibre density (CNFD)—nerve fibres/mm² of corneal tissue in subjects with hyperlipidaemia prior to treatment (baseline) and six months after treatment with a PCSK9 monoclonal antibody (6 months) compared to age and sex matched healthy control subjects

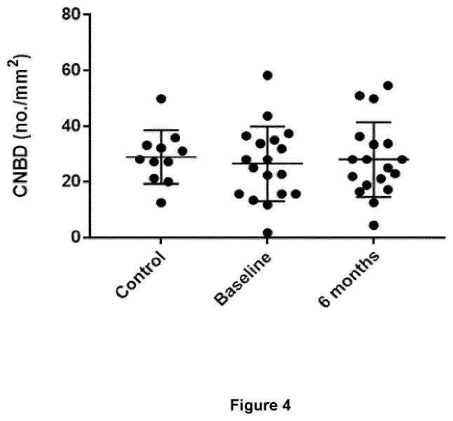

FIG. 4 is a graph showing changes in corneal nerve branch density (CNBD)—nerve branches/mm² of corneal tissue in subjects with hyperlipidaemia prior to treatment (baseline) and six months after treatment with a PCSK9 monoclonal antibody (6 months) compared to age and sex matched healthy control subjects; and

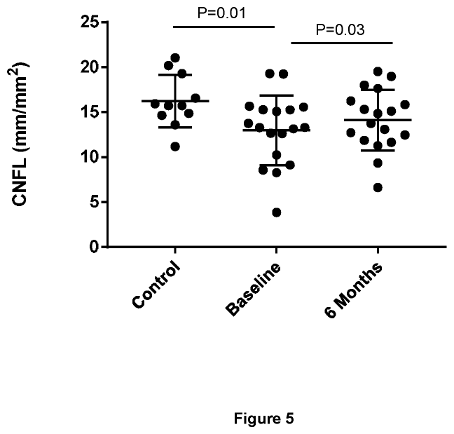

FIG. 5 is a graph showing changes in corneal nerve fiber length (CNFL)—length of nerves/mm² of corneal tissue in subjects with hyperlipidaemia prior to treatment (baseline) and six months after treatment with a PCSK9 monoclonal antibody (6 months) compared to age and sex matched healthy control subjects;

FIG. 6 shows a series of graphs which show that Corneal small nerve fibre parameters correlate with total (black dots) and free (grey triangles) serum PCSK9;

FIG. 7 shows a series of graphs which shows changes in nerve parameters from subjects with Heterozygous Familial Hypercholesterolemia (HeFH) with no history of diabetes (DM) at baseline compared to matched controls;

FIG. 8 shows a series of graphs which show the effect of treating subjects with Heterozygous Familial Hypercholesterolemia (HeFH) with no history of diabetes (DM) at baseline and followed up 12 months after treatment with PCSK9 monoclonal antibodies; and

Table 1 shows clinical and metabolic characteristics in healthy control and morbidly obese subjects;

Table 2 shows values obtained using different tests for neuropathy in healthy control and morbidly obese subjects;

Table 3 shows expression values for various markers in the blood of samples from morbidly obese subjects with small fibre neuropathy versus morbidly obese subjects without small fibre neuropathy; and

Table 4 shows that PCSK9 expression levels correlate with other tests and markers of neuropathy;

Table 5 shows the correlation of Corneal small nerve fibre parameters with total and free serum PCSK9;

Table 6 shows a nerve parameters from subjects with Heterozygous Familial Hypercholesterolemia (HeFH) with no history of diabetes (DM) at baseline compared to age and gender matched controls; and

Table 7 shows nerve parameters from subjects with Heterozygous Familial Hypercholesterolemia (HeFH) with no history of diabetes (DM) at baseline and followed up 12 months after treatment with PCSK9 monoclonal antibodies.

### EXAMPLES

Materials and Methods

Subject Recruitment

Human subjects with HeFH (heterozygous familial hypercholesterolemia) were recruited from Manchester Royal Infirmary's Lipid Clinic and compared with 11 age and sex matched healthy volunteer controls.

For example 2, human subjects with morbid obesity attending a tertiary centre weight management clinic, were recruited from Salford Royal Hospital and compared with 20 healthy volunteers. Exclusion criteria were history of cancer, previous chemotherapy or radiotherapy, history of corneal trauma or surgery or a history of ocular or systemic disease that may affect the cornea. Patients receiving treatment with lipid modifying agents were excluded. The study was approved by the Research and Ethics Committee, and written informed consent was obtained from all subjects prior to participation. This research adhered to the declaration of Helsinki and all subjects provided written informed consent.

Demographics and Assessment of Neuropathy

All study participants underwent assessment of body mass index (BMI (kg/m²) and blood pressure (Dinamap pro 100v2, GE Medical Systems, Freiburg, Germany). Neuropathic symptoms were assessed using the Neuropathy Symptom Profile (NSP) and neurological deficits were evaluated using the modified neuropathy disability score (NDS). Vibration perception threshold (VPT) was tested using a Neurothesiometer (Horwell, Scientific Laboratory Supplies, Wilfrod, Nottingham, UK). Cold (CT) and warm (WT) thresholds were assessed on the dorsolateral aspect of the left foot (S1) using the TSA-II NeuroSensory Analyser (Medoc Ltd., Ramat-Yishai, Israel). Electro-diagnostic studies were undertaken using a Dantec “Keypoint” system (Dantec Dynamics Ltd, Bristol, UK) equipped with a DISA temperature regulator to keep limb temperature constantly between 32-35° C. Sural sensory nerve amplitude, conduction velocity and latency and peroneal motor nerve amplitude, conduction velocity and latency were assessed by a consultant neurophysiologist. The motor nerve study was performed using silver-silver chloride surface electrodes at standardized sites defined by anatomical landmarks and recordings for the sural sensory nerve was taken using antidromic stimulation over a distance of 100 mm. Heart rate variability to deep breathing (HRV-DB) was assessed with an ANX 3.0 autonomic nervous system monitoring device (ANSAR Medical Technologies Inc., Philadelphia, Pa., USA).

Corneal Confocal Microscopy

Patients underwent examination with a CCM (Heidelberg Retinal Tomograph III Rostock Cornea Module, Heidelberg Engineering GmbH, Heidelberg, Germany) as per our previously established protocol (25). Six non-overlapping images/patient (3 per eye) from the centre of the cornea were selected and quantified in a masked fashion. Automated analysis of corneal nerve morphology was performed using ACCMetrics. Corneal nerve fiber density (CNFD)—the total number of major nerves/mm² of corneal tissue, Corneal nerve branch density (CNBD)—the number of branches emanating from the major nerve trunks/mm² of corneal tissue and Corneal nerve fiber length (CNFL)—the total length of all nerve fibres and branches (mm/mm²) within the area of corneal tissue were quantified.

Blood Sampling

After collection of fasting blood samples, serum and EDTA-plasma were isolated by centrifugation at 2000 g for 15 minutes at 4° C. within 2 hours of collection and stored until further use. Total cholesterol was measured using the cholesterol oxidase phenol 4-aminoantipyrine peroxidase method, triglycerides by the glycerol phosphate oxidase phenol 4-aminoantipyrine peroxidase method. HDL cholesterol was assayed using the direct clearance method. All these tests were performed on a RX daytona analyzer (Randox Laboratories Ltd, Crumlin, County Antrim, UK). The laboratory participated in RIQAS (Randox International Quality Assessment Scheme; Randox Laboratories, Dublin, Ireland), which is CRC calibrated. LDL cholesterol was estimated using the Friedewald formula. OxLDL was measured in plasma by a direct ELISA sandwich technique with a kit from Mercodia, Upsala, Sweden. Intra-assay and inter-assay coefficient of variance (CV) were 3.4% and 14.9%. PCSK9 was measured using an ELISA (R&D Systems Europe, Abingdon, UK) which was based on the antibody sandwich principle with intra-assay and inter-assay coefficient of variance (CV) of less than 8%.

Statistical Analysis

Analysis was carried out on SPSS for Mac (Version 19.0, IBM Corporation, New York, USA). All data are expressed as mean±standard error of mean (SEM). The data were assessed for normality and appropriate statistical analyses conducted. To assess within and between group differences we used one-way analysis of variance (ANOVA) or a non-parametric counterpart (Kruskal-Wallis). A significant p value was considered to be <0.05. Post hoc analyses (Tukey) maintained an overall p value of 0.05 for significance.

Separation of Lipoprotein Fractions

Lipoprotein fraction separation was done using immunoprecipitation reagent kit from Sun Diagnostics, LLC (LipoSep IP™, Sun Diagnostics). All apoB-containing lipoproteins were precipitated using stabilised goat anti-apoB sera. Equal volumes of serum and the IP reagent were vortexed and incubated for 10 minutes at room temperature, and then centrifuged for 10 min at 12000 rpm in a bench-centrifuge (Sigma laborzentrifugen sigma 3-16K). The supernatant was then collected into micro tubes for measurement of PCSK9.

PCSK9 Measurement

Measurement of PCSK9 concentration was undertaken using a sandwich ELISA kit (R&D Systems DY3888). Briefly, 96-well plates were coated with 100 μl polyclonal rat anti-human PCSK9 antibody and incubated overnight at room temperature. The plate was washed twice with PBS and blocked with 150 μl of PBS containing 1% bovine serum albumin (BSA) for 1 hour at room temperature. After four washes with PBS containing 0.1% Tween 20, 100 μl samples or standards were added and incubated at room temperature for 2 hours. This is followed by four washes, addition of 100 μl biotinylated sheep anti-human PCSK9 detection antibody, and a further 2 hours of incubation at room temperature. The plate was then washed a further four times and 100 μl streptavidin HRP was added and incubated for 20 minutes at room temperature. After another four washes, 100 μl of 3,3′,5,5′-Tetramethylbenzidine substrate (Life Technologies Ltd) was added and incubated at room temperature for 30 min. Colour development was stopped using 50 μl of stop solution (2N H₂SO₄) and absorbance read at 450 nm using ELISA reader (BioTek Instruments, Inc EL800).

Determination of Total and Non-apoB100-Containing Lipoprotein-Bound PCSK9

Total PCSK9 concentration was determined using the above method in both serum and plasma of participants. PCSK9 was measured in the supernatant following apoB-LP depletion by immunoprecipitation.

### Example 1

Treatment of Patients with a PCSK9 Monoclonal Antibody Increases CNFD, CNBD and CNFL

18 human subjects with heterozygous familial hypercholesterolemia (HeFH) were treated with a PCSK9 monoclonal antibody. The corneal nerves in the cornea of the patients were photographed using a corneal confocal microscope (CCM). Images were obtained from the subject prior to the start of treatment (baseline, B), from the same subject after six months of treatment with a PCSK9 monoclonal antibody (C) and also compared to healthy age and gender matched controls (A). Example CCM images from are shown in FIG. 1. This Figure shows that treatment with the PCSK9 monoclonal antibody increased the number of nerves in the cornea of the subject.

Human subjects with diabetes were also treated with a PCSK9 monoclonal antibody. The corneal nerves in the cornea of the patients were photographed using a corneal confocal microscope (CCM). Example images are shown in FIG. 2. Images were obtained from the subject prior to the start of treatment (baseline, A) and from the same subject after six months of treatment with a PCSK9 monoclonal antibody (B). Corneal nerve fibre length (CNF) increased in the patient following six months of treatment (baseline 25.89 to 28.12 mm/square mm).

Corneal confocal microscopy was then used to determine the CNFD, CNBD and CNFL of subjects with hyperlipidaemia prior to treatment and following six months of treatment with a PCSK9 monoclonal antibody. The CNFD, CNBD and CNFL of 11 age and sex matched healthy controls were also measured using CCM images.

FIGS. 3 to 5 show the quantified levels of CNFD, CNBD and CNFL. All data is presented as Mean±SD.

At the baseline level, the CNFD of the hyperlipidaemia subjects was significantly lower (P=0.002) than the CNFD of healthy age and sex matched controls (baseline value of 20.58±7.1020.58 compared to 29.13±6.11 of the control) (FIG. 3). Following six months of treatment, the CNFD of the hyperlipidaemia subjects significantly increased from baseline levels (P=0.01) to an average of 23.12±5.69 (FIG. 3).

A similar trend was observed with CNBD. At the baseline level, the mean CNBD of the hyperlipidaemia subjects was lower than the mean CNBD of the healthy controls (26.51±13.48 versus 28.98±9.72) (FIG. 4). However, following treatment the mean CNBD of the hyperlipidaemia subjects was increased to nearly equal levels to those of the healthy controls (28.01±13.5 versus 28.98±9.72) (FIG. 4).

The CNFL of the hyperlipidaemia subjects prior to treatment at the baseline level had a mean value of 12.99±3.87. This was significantly lower (P=0.01) than the CNFL of the healthy control group (16.23±2.9). Following treatment, the CNFL in the hyperlipidaemia subjects had significantly increased (P=0.03) to 14.10±3.38 (FIG. 5).

These results indicate that PCSK9 antibodies can be used to treat neuropathy.

### Example 2

Morbidly Obese Subjects have a Higher Level of PCSK9 Expression than Non-Obese Control Subjects

The clinical and metabolic characteristics comparing control and morbidly obese subjects are shown in Table 1. Obese subjects had a significantly higher BMI (P<0.001), HbA1c (P=0.001), triglycerides (P=0.03) and CRP (P<0.001) with a lower total cholesterol (P=0.04), LDL cholesterol (P=0.04) and HDL (P<0.001). Obese subjects had a significantly higher level of PCSK9 compared to controls (P=0.01).

CCM and Other Techniques can be Used to Diagnose Neuropathy in Subjects

Typical tests for the diagnosis of neuropathy include the Neuropathy Symptom Profile (NSP), the modified Neuropathy Disability Score (NDS), the vibration perception threshold (VPT), cold (CT) and warm (WT), sural sensory nerve amplitude, conduction velocity and latency and peroneal motor nerve amplitude, conduction velocity and latency and heart rate variability to deep breathing (HRV-DB).

A plurality of these tests are typically used in combination to assess and diagnose neuropathy in a subject.

The results of these tests are shown in Table 2. NSP (p<0.001) and VPT (p<0.001) were higher and sural nerve amplitude (p=0.001), peroneal nerve amplitude (p=0.02) and peroneal nerve conduction velocity (p=0.05) were significantly lower in the morbidly obese subjects compared to controls. WT (p=0.003) was increased, whilst CT (p=0.02) and HRV-DB (p=0.002) were decreased in morbidly obese subjects relative to healthy control subjects.

These values indicate that the morbidly obese subjects had significant large and small fibre neuropathy compared to controls.

CNFD, CNBD and CNFL were also measured using corneal confocal microscopy of the morbidly obese and the healthy control subjects. It was observed that levels of CNFD, CNBD and CNFL were each significantly reduced (p=0.001) in morbidly obese subjects relative to healthy control subjects. This reduction in levels corresponded to the increase or decrease observed using the other known parameters for neuropathy diagnosis. This indicates that CNFD, CNBD and/or CNFL can be used to diagnose neuropathy in a subject.

14/34 morbidly obese subjects had a small fibre neuropathy based on a CNFL cut off of <2SD of control subjects.

PCSK9 Expression Levels in Peripheral Blood are Higher in Morbidly Obese Subjects with Small Fibre Neuropathy

Morbidly obese subjects with small fibre neuropathy as diagnosed in Table 2 compared to morbidly obese subjects without small fibre neuropathy showed no difference in age, BMI, waist circumference, HbA1c, triglycerides, total cholesterol, triglycerides, HDL cholesterol, oxLDL or CRP. However, LDL-C and PCSK9 levels were significantly higher in morbidly obese subjects with small fibre neuropathy compared to those without small fibre neuropathy (Table 3).

PCSK9 levels correlated significantly with CNFD (r=−0.54, p=0.003), CNBD (r=−0.49, p=0.007) and CNFL (r=−0.56, p=0.002) (Table 4). PCSK9 also correlated significantly with HRV-DB (r=0.5, p=0.05). There was no significant correlation between different lipoproteins and CCM small nerve fibre assessments (Table 4).

Multiple regression analysis revealed that PCSK9 was associated with CNFL (p=0.03), independently of HbA1c, triglycerides, LDL-C, HDL-C and oxLDL

This indicates that PCSK9 can be used as a marker of neuropathy independently of other markers. It also suggests that inhibition of PCSK9 expression can be used as a treatment of neuropathy.

### Example 3

Familial Hypercholesterolemia (FH) with No History of Diabetes (DM) Compared to Controls at Baseline and Followed Up 12 Months after Treatment with PCSK9 Monoclonal Antibodies

Study 1: Cross-Sectional Study Method

Objective

To determine if there is a role for PCSK9 in the pathogenesis of obesity related neuropathy and the role of total and free PCSK9.

Research Design and Methods

Twenty-nine morbidly obese participants and twenty control (not on glucose or lipid lowering medications) and 20 healthy controls underwent detailed assessment of neuropathy and circulating levels total and free PCSK9 and lipoproteins.

Results

Obese non-diabetic patients had large and small nerve fibre neuropathy. Total serum PCSK9 is higher in the obese group compared to controls. Among the obese group, those with evidence of neuropathy has a significantly higher circulating total serum PCSK9 level. Among the obese group, free and total serum PCSK9 inversely correlated strongly with Corneal Nerve fibre Length (CNFL), Corneal Nerve Fibre Density (CNFD), Corneal Nerve Branch Density (CNBD) and heart rate variability (HRV) (FIG. 6 and table 4)

Study 2: Heterozygous Familial Hypercholesterolemia (HeFH) with No History of Diabetes (DM) at Baseline Compared to Matched Controls and Effect of Targeting PCSK9 Therapeutically—Interm Analysis

Objective To assess if patients with Heterozygous Familial Hypercholestrolemia (HeFH) has nerve fibre damage compared to matched controls and if treatment with PCSK9 monoclonal antibodies improves small nerve structure in an open label single arm study

Methods We recruited 44 patients known to have HeFH and 20 matched controlled. We followed HeFH patients and reassessed them 12 months after treatment with one of the PCSK9 monoclonal antibodies [Evolocumab (70% and Alirocumab (30%)]. All HeFH patients undergone corneal confocal microscopy to assess corneal nerve fibre integrity and structure and healthy controls at baseline.

Results

At baseline: patients with HeFH had a significant corneal nerve damage (small fire neuropathy) (FIG. 7 and table 5).

We followed the HeFH patients and reassessed them 12 months after treatment with PCSK9 monoclonal antibodies:

Corneal nerve fibre length (CNFL), corneal nerve fibre density (CNFD) and corneal nerve branch density (CNBD) improved significantly (please see table 6 and FIG. 8).

Further Work

Initial pilot studies have been carried out on 9 patients with type 2 diabetes and after 6 months of treatment with anti-PCSK9 antibodies, an improvement in corneal nerves was observed.

Conclusions

Total and free serum PCSK9 levels robustly and constantly correlates with all corneal nerve fibre parameters (CNFL, CNFD and CNFD).

High free serum PCSK9 levels associated with small nerve fibre damage

High total serum PCSK9 levels associated with small nerve fibre damage

The correlation seems to be stronger between CNFL and serum free PCSK9.

The above indicate a direct involvement of PCSK9 in neuropathy and nerve damage rather than operating via lipid parameters like LDL particles or LDL cholesterol.

Patients with HeFH (no diabetes) have small nerve fibre damage when compared with healthy controls.

Small nerve fibre damage improves, with evidence of regeneration, in FH patients 12 months after treatment with PCSK9 monoclonal antibodies.

This indicates that nerve fibre regenerates after targeting circulating PCSK9 with PCSK9 monoclonal antibodies.

TABLE 1
Demographic and lipid data for control vs obese participants.
^{*}P < 0.05, \hat{ }P < 0.005, ⁺P < 0.001

| | | | No SFN | SFN |
|---|---|---|---|---|
| | Control | Obese | (n = 20) | (n = 14) |
| Age (Years) | 45.5 ± 2.5 | 45.5 ± 1.6 | 45.5 ± 1.8 | 47.3 ± 2.5 |
| BMI (kg/m²) | 25.3 ± 1.0 | 48.4 ± 1.4⁺ | 49.3 ± 1.8 | 48.1 ± 2.5 |
| IFCC (mmol/mol) | 37.4 ± 0.7 | 46.1 ± 2.9\hat{ } | 44.7 ± 3.4 | 47.4 ± 5.2 |
| Cholesterol (mmol/l) | 4.9 ± 0.2 | 4.4 ± 0.2^{*} | 4.4 ± 0.2 | 4.3 ± 0.3 |
| Triglyceride (mmo1/l) | 1.1 ± 0.2 | 1.7 ± 0.2^{*} | 1.4 ± 0.2 | 2.0 ± 0.4 |
| Direct HDL (mmol/l) | 1.4 ± 0.1 | 1.0 ± 0.1⁺ | 1.1 ± 0.1 | 0.9 ± 0.1 |
| LDL-C (mmol/l) | 3.0 ± 0.1 | 2.6 ± 0.1^{*} | 2.4 ± 0.2 | 2.7 ± 0.2^{*} |
| oxLDL (U/l) | 40.0 ± 2.1 | 39.5 ± 1.8 | 40.9 ± 2.5 | 38.8 ± 2.4 |
| CRP (mg/l) | 2.5 ± 0.8 | 7.7 ± 1.0⁺ | 8.4 ± 1.6 | 7.6 ± 1.4 |
| PCSK9 (ng/ml) | 672.9 ± 27.8 | 927.5 ± 68.8^{*} | 833.2 ± 77.9 | 1096.7 ± 108.5^{*} |

TABLE 2
Neuropathy assessments for control vs obese participants.
^{*}P < 0.05, \hat{ }P < 0.005, ⁺P < 0.001

| | Control | Obese |
|---|---|---|
| | (n = 20) | (n = 34) |
| NSP | 0.4 ± 0.2 | 4.7 ± 1.0⁺ |
| NDS | 0.3 ± 0.1 | 1.2 ± 0.4 |
| VPT (volts) | 4.3 ± 0.5 | 10.4 ± 1.2⁺ |
| Sural amplitude (μV) | 20.1 ± 1.5 | 12.7 ± 1.5\hat{ } |
| Sural velocity (m/s) | 51.0 ± 0.8 | 48.6 ± 1.6 |
| Peroneal amplitude | 5.5 ± .04 | 4.2 ± 0.4^{*} |
| (mV) | | |
| Peroneal velocity (m/s) | 48.8 ± 0.6 | 46.2 ± 1.0^{*} |
| CT (° C.) | 28.2 ± 0.5 | 25.9 ± 1.0^{*} |
| WT (° C.) | 37.3 ± 0.5 | 40.1 ± 0.6\hat{ } |
| DB-HRV (beats per min) | 31.1 ± 2.3 | 21.5 ± 2.5\hat{ } |
| CNFD (no/mm²) | 31.4 ± 1.4 | 25.6 ± 1.0\hat{ } |
| CNBD (no/mm²) | 39.3 ± 3.0 | 32.3 ± 2.8^{*} |
| CNFL (mm/mm²) | 17.9 ± 0.7 | 14.8 ± 0.5\hat{ } |

TABLE 3
Demographic and lipid data for participants
with and without small fibre
neuropathy.^{*}P < 0.05

| | No SFN | SFN |
|---|---|---|
| | (n = 20) | (n = 14) |
| Age (Years) | 45.5 ± 1.8 | 47.3 ± 2.5 |
| BMI (kg/m²) | 49.3 ± 1.8 | 48.1 ± 2.5 |
| IFCC (mmol/mol) | 44.7 ± 3.4 | 47.4 ± 5.2 |
| Cholesterol (mmol/l) | 4.4 ± 0.2 | 4.3 ± 0.3 |
| Triglyceride (mmol/l) | 1.4 ± 0.2 | 2.0 ± 0.4 |
| Direct HDL (mmol/l) | 1.1 ± 0.1 | 0.9 ± 0.1 |
| LDL-C (mmol/l) | 2.4 ± 0.2 | 2.7 ± 0.2^{*} |
| oxLDL (U/l) | 40.9 ± 2.5 | 38.8 ± 2.4 |
| CRP (mg/l) | 8.4 ± 1.6 | 7.6 ± 1.4 |
| PCSK9 (ng/ml) | 833.2 ± 77.9 | 1096.7 ± 108.5^{*} |

TABLE 4
Cross-sectional study: Corneal small nerve fibre parameters correlation with total and free PCSK9 serum.

| | PCSK9 | HbA1c | Cholesterol | HDL | LDL | Triglycerides |
|---|---|---|---|---|---|---|
| Sural Amplitude (μV) | r = −0.3, p = 0.3 | r = 0.3, p = 0.2 | r = −0.8, p = 0.7 | r = −0.1, p = 0.7 | r = −0.2, p = 0.4 | r = 0.3, p = 0.3 |
| Sural Velocity (m/s) | r = 0.1, p = 0.8 | r = 0.002, p = 0.9 | r = −0.01, p = 0.9 | r = −0.1, p = 0.8 | r = −0.02, p = 0.8 | r = 0.03, p = 0.8 |
| Peroneal | r = −0.3, p = 0.3 | r = −0.2, p = 0.3 | r = 0.03, p = 0.9 | r = −0.0, p = 0.9 | r = 0.1, p = 0.6 | r = −0.1, p = 0.5 |
| amplitude (mV) | | | | | | |
| Peroneal | r = −0.1, p = 0.8 | r = −0.4, p = 0.6 | r = −0.3, p = 0.2 | r = −0.3, p = 0.2 | r = −0.1, p = 0.5 | r = −0.1, p = 0.6 |
| Velocity (m/s) | | | | | | |
| CT (° C.) | r = −0.2, p = 0.3 | r = 0.1, p = 0.6 | r = 0.2, p = 0.3 | r = 0.04, p = 0.8 | r = 0.1, p = 0.5 | r = 0.1, p = 0.5 |
| WT (° C.) | r = 0.2, p = 0.5 | r = −0.2, p = 0.4 | r = 0.05, p = 0.8 | r = 0.3, p = 0.1 | r = 0.01, p = 0.9 | r = −0.1, p = 0.6 |
| HRV (beats per min) | r = 0.5, p = 0.05 | r = −0.2, p = 0.4 | r = −0.2, p = 0.5 | r = 0.2, p = 0.5 | r = −0.004, p = 0.9 | r = −0.3, p = 0.2 |
| CNFD (no/mm²) | r = −0.4, p = 0.02 | r = −0.1, r = 0.5 | r = −0.2, p = 0.2 | r = 0.02, p = 0.9 | r = 0.1, p = 0.5 | r = −0.2, p = 0.3 |
| CNBD (no/mm²) | r = −0.4, p = 0.04 | r = −0.4, p = 0.4 | r = −0.07, p = 0.7 | r = 0.03, p = 0.9 | r = −0.07, p = 0.7 | r = −0.02, p = 0.7 |
| CNFL (mm/mm²) | r = −0.4, p = 0.03 | r = −0.11, p = 0.9 | r = −0.2, p = 0.3 | r = 0.04, p = 0.8 | r = −0.09, p = 0.6 | r = −0.2, p = 0.2 |

TABLE 5
Cross-sectional study: Corneal small nerve fibre parameters
correlation with total and free PCSK9 serum

| | CNFD | CNBD | CNFL | HRV |
|---|---|---|---|---|
| PCSK9 (ng/ml) | r = −0.37, P = 0.04 | r = −0.38, P = 0.04 | r = −0.3, P = 0.1 | r = 0.5, p = 0.04 |
| Free PCSK9 (ng /ml) | r = −0.42, P = 0.02 | r = −0.36, P = 0.05 | r = −0.34, P = 007 | |

CNFD; Corneal Nerve Fibre Density
CNBD; Corneal Nerve Branch Density
CNFL; Corneal Nerve Fibre Length
HRV; Heart rate Variability

TABLE 6
Heterozygous Familial Hypercholesterolemia
(HeFH) with no history of diabetes (DM) at baseline
compared to age and gender matched controls

| | Healthy | Baseline | P value |
|---|---|---|---|
| | controls | patients | controls |
| Parameters | (N = 20) | (N = 44) | vs patients |
| Age (years) | 52.25 ± 12.38 | 56.45 ± 11.33 | 0.2 |
| CNFD (no/mm²) | 31.13 ± 6.78 | 23.30 ± 5.14 | <0.0001 |
| CNBD (no/mm²) | 41.55 ± 14.94 | 27.12 ± 12.07 | <0.0001 |
| CNFL (mm/mm²) | 17.62 ± 2.94 | 13.85 ± 2.48 | <0.0001 |

TABLE 7
Heterozygous Familia Hypercholesterolemia (HeFH) with
no history of diabetes (DM) at baseline and followed up 12
months after treatment with PCSK9 monoclonal antibodies

| | | 12 months | |
|---|---|---|---|
| | Baseline | follow | P value |
| | patients | up patients | baseline vs |
| Parameters | (N = 44) | (N = 44) | follow up |
| CNFD (no/mm²) | 23.30 ± 5.14 | 27.63 ± 6.68 | <0.0001 |
| CNBD (no/mm²) | 27.12 ± 12.07 | 31.03 ± 13.88 | 0.03 |
| CNFL (mm/mm²) | 13.85 ± 2.48 | 15.64 ± 3.09 | <0.0001 |

^{●} Results are expressed as mean ± SD,

1. A method of treating peripheral and/or autonomic neuropathies in a subject in need thereof, the method comprising administering to the subject an agent which is capable of inhibiting PCSK9.

2. The method according to claim 1 wherein the agent inhibits the activity of PCSK9.

3. The method according to claim 1, wherein the agent comprises an antibody.

4. The method according to claim 3, wherein the agent comprises a monoclonal antibody.

5. The method according to claim 4, wherein the monoclonal antibody is selected from evolocumab and alirocumab.

6. The method according to claim 1, wherein the neuropathy is diabetic neuropathy.

7. The method according to claim 1, wherein the subject has type 2 diabetes.

8. The method according to claim 1, wherein the subject has a BMI (body mass index) of 25 or more.

9. The method according to claim 8, wherein the subject has a BMI of 30 or more.

10. The method according to claim 8, wherein the subject has a BMI of 40 or more.

11. The method according to claim 1, wherein the neuropathy is retinopathy.

12. The method according to claim 1, wherein the neuropathy is small fibre neuropathy.

13. The method according to claim 1, wherein the method comprises administering the agent to the subject by intravenous, intramuscular or intradermal injection.

14. The method according to claim 1, wherein the method comprises administering the agent to the subject once a fortnight or once a month.

15. The method according to claim 1, wherein the method comprises administering the agent to the subject at a dose of between 130 and 450 mg.

16. The method according to claim 1 wherein the method comprises administering the agent to the subject at a dose of 140 mg.

17. The method according to claim 1, wherein the method comprises administering the agent with an LDL cholesterol lowering medication.

18. The method according to claim 17, wherein the LDL-lowering cholesterol medication comprises a statin.

19. The method according to claim 1, wherein the agent increases corneal nerve fibre density, corneal nerve fibre length and/or corneal nerve branch density in the subject.

20. A method of detecting a level of PCSK9 expression in a subject in order to detect whether or not the subject is suffering from a neuropathy, such as peripheral and/or autonomic neuropathy, the method comprising:
determining a level of PCSK9 expression in a sample obtained from the subject, wherein the level of PCSK9 is total or free serum PCSK9 and an elevated level of PCSK9 is correlated with the subject suffering from neuropathy.

21. The method according to claim 20, wherein the sample is a fluid sample.

22. The method of claim 20, wherein the sample comprises plasma.
